(19) United States
(12) Patent Application Publication
(10) Pub. No.: US 2021/0179543 A1
(43) Pub. Date: Jun. 17, 2021

(54) CALPAIN-2 SELECTIVE INHIBITOR COMPOUNDS FOR TREATMENT OF GLAUCOMA

(71) Applicant: Western University of Health Sciences, Pomona, CA (US)

(72) Inventors: Michel Baudry, Corona, CA (US); Yun Luo, Pomona, CA (US); Norton P. Peet, Holland, MI (US)

(21) Appl. No.: 17/267,802

(22) PCT Filed: Aug. 13, 2019
(86) PCT No.: PCT/US2019/046431

§ 371 Date: Feb. 10, 2021

Related U.S. Application Data
(60) Provisional application No. 62/718,088, filed on Aug. 13, 2018.

(51) Int. Cl. C07C 237/22; A61P 27/06; A61K 9/00

(52) U.S. Cl. CPC C07C 237/22; A61K 9/0048; A61P 27/06

(57) ABSTRACT

Compounds of Formula (I) are provided including for treatment of disorders such as glaucoma.

### CROSS-REFERENCE TO RELATED APPLICATIONS

This application claims the benefit of U.S. Provisional Application No. 62/718,088 filed Aug. 13, 2018, the entire contents of which are incorporated by reference herein.

### FIELD OF THE INVENTION

In one aspect, the invention relates to compounds for the treatment of glaucoma including acute glaucoma or other acute eye disorders, pharmaceutical compositions comprising said compounds, and methods of treating glaucoma with said compounds.

### BACKGROUND

Glaucoma is a disease of the optic nerve and the elevated eye pressures are related to damage of this nerve. The optic nerve carries images from the retina to the brain. Glaucoma damages optical nerve cells causing blindspots to occur within a subject's vision. These blind spots typically are not noticed by the subject until considerable damage to the optic nerve has already occurred. The terminal stage of glaucoma is total blindness of the subject.

The two major calpain isoforms in the brain, calpain-1 and calpain-2, play opposite functions in both synaptic plasticity and neurodegeneration. While calpain-1 is required for the induction of synaptic plasticity, calpain-2 limits the extent of synaptic plasticity during the minutes following the induction event (Wang, Y. et a!. A molecular brake controls the magnitude of long-term potentiation. Nat Commun 5, 3051, (2014); likewise, calpain-1 is neuroprotective and calpain-2 is neurodegenerative (Wang et al., J. Neuro. 27 Nov. 2013, 33 (48) 18880-18892). These dual and opposite functions of calpain-1/2, as well as the lack of selective inhibitors for these two calpain isoforms account for the previous difficulties in developing calpain inhibitors for translational applications, and in particular for preventing neurodegeneration. Calpain-1 activation is linked to synaptic NMDA receptor stimulation, which accounts for its necessary role in long term potentiation (LTP) induction. It is also involved in neuroprotection elicited by synaptic NMDA receptor stimulation. On the other hand, calpain-2 is linked to extrasynaptic NMDA receptor stimulation and is involved in neurodegeneration. Calpain-2 is also activated by BDNF->ERK-mediated phosphorylation and limits the extent of LTP following theta-burst stimulation (TBS). Thus, a selective calpain-2 inhibitor can be both neuroprotective and a cognitive enhancer. Selective calpain-2 inhibitors could be used for a number of acute indications associated with neuronal death, including stroke, concussion, intracerebral hemorrhage, acute glaucoma, and spinal cord injury.

International Patent Application No. PCT/US2015/060157, describes isoform-specific calpain inhibitors, methods of identification, and uses thereof. Examples of inhibitors exhibiting higher selectivity for one calpain versus another have been disclosed (Li, Z. et a!. Novel peptidyl α-keto amide inhibitors of calpains and other cysteine proteases. Journal of medicinal chemistry 39, 4089-4098 (1996); Li, Z. et al. Peptide. α-keto ester, α-keto amide, and α-keto acid inhibitors of calpains and other cysteine proteases. Journal of medicinal chemistry 36, 3472-3480 (1993)). However, these studies acknowledged that the usefulness of a calpain-1 or calpain-2-selective inhibitor was unknown and required additional experimentation to determine if these compounds actually had therapeutic value.

A selective calpain-2 inhibitor, Z-Leu-Abu-CONH-CH2-C6H3(3,5-(OMe)2, (“C2I”) which both enhances learning and is neuroprotective has been previously identified. (Wang, Y. et al. A molecular brake controls the magnitude of long-term potentiation. Nat Commun 5, 3051, (2014); Liu, Y. et al. A calpain-2 selective inhibitor enhances learning & memory by prolonging ERK activation. Neuropharmacology 105, 471-477, doi:10.1016/j.neuropharm.2016.02.022 (2016). See also Wang, et al., (2016) Neurobiol Dis. 2016 September; 93:121-8.

It would be desirable to have additional calpain inhibitors, including calpain-2 inhibitors.

### SUMMARY OF THE INVENTION

In one aspect, compounds which are selective inhibitors of calpain-2 are provided.

Preferred compounds can be useful to treat acute glaucoma. Preferred compounds also may be useful to treat various eye disorders associated with retinal neuronal cell death.

In a particular aspect, compounds of the following Formula (I) are provided:

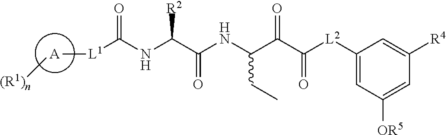

wherein A is carbocyclic aryl or heteroaryl

R¹ is a non-hydrogen substituent such as C₁₋₆alkyl, halogen, cyano, nitro, C₁₋₆alkoxy;

n is an integer of from 0 (where the ring A is unsubstituted) to the value permitted by the valence of the ring such as 5 where A is phenyl;

L¹ and L² are each the same or different optionally substituted alkylene having one to 6 carbons (e.g. —(CH₂)ₙ where n is 1 to 6 and each carbon may have zero, one or two non-hydrogen substituents),

R² is non-hydrogen substituent such as optionally substituted C₁₋₆alkyl,

R⁴ is hydrogen or halogen such as fluoro; R⁵ is C₁₋₆alkyl such as methyl; and pharmaceutically acceptable salts thereof.

In certain preferred aspects, R⁴ is hydrogen or fluoro and R⁵ is methyl. In a particular aspect, R⁴ is fluoro and R⁵ is methyl. In another particular aspect, R⁴ is hydrogen and R⁵ is methyl.

In preferred aspects, one or both of L¹ and L² are unsubstituted alkylene such as methylene (—CH₂—).

In additional preferred aspects, the group A is carbocyclic aryl such as phenyl or a heteoraryl with one of more nitrogen ring members such as optionally substituted pyridinyl or optionally substituted pyrazinyl.

In certain aspects, n may be 0, 1, 2, or 3, such as 0 or 1, or 0.

In particularly preferred aspects, the following compound 17 and compound 15 are provided:

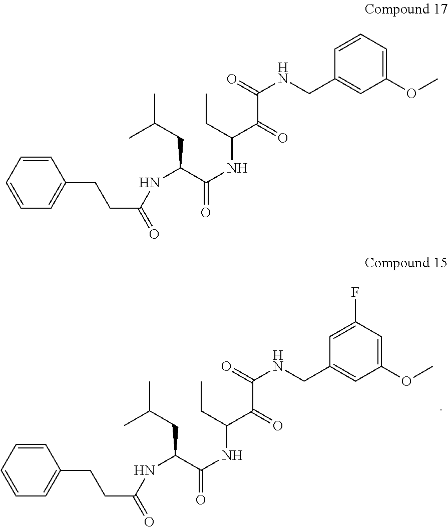 [SMILES: CCC(NC(=O)[C@H](CC(C)C)NC(=O)CCc1ccccc1)C(=O)C(=O)NCc1cccc(OC)c1 ; CCC(NC(=O)[C@H](CC(C)C)NC(=O)CCc1ccccc1)C(=O)C(=O)NCc1cc(F)cc(OC)c1]

Pharmaceutical compositions comprising said compounds, and methods of treating glaucoma with said compounds are also provided. In particular aspects, methods are provided for treating a subject suffering from or susceptible to an eye or ocular disease or disorder including for example glaucoma, including open-angle glaucoma, angle-closure glaucoma, normal tension glaucoma, congenital glaucoma, pigmentary glaucoma, pseudoexfoliative glaucoma, traumatic glaucoma, neovascular glaucoma, irido corneal endothelial syndrome, ischemia in the eye, and/or ischemia in the retina.

Methods of treatment is general comprise administering to a subject such as a mammal, particularly primate including a human, an effective amount of one or more compounds as disclosed herein. A subject suitably may be identified and selected for treatment. For instance, the subject may be identified as suffering from a particular disease or disorder such as an eye or ocular disorder for example glaucoma. The one or more compounds disclosed herein then may be administered to the identified subject.

In additional aspects, the present compounds may be utilized for treatment of various diabetes disorders. In particular aspects, a subject suffering from Wolfram syndrome including Wolfram syndrome 1 or Wolfram syndrome 2 may be treated.

As discussed further below, we have demonstrated intra-ocular injection of selective calpain-2 inhibitors in an in vivo glaucoma model. Such compounds may be used for the treatment of a variety of eye disorders associated with neuronal death in the retina.

Other aspects of the invention are disclosed infra.

### BRIEF DESCRIPTION OF THE DRAWINGS

FIG. 1 shows an analog of C2I (a.k.a. NA101), compound 15 (a.k.a. NA115), dose-dependently inhibiting calpain-2 activation in the retina following increased IOP. Immunohistochemistry for SBDP (green) in sections from a sham animal (surgery only), animal subjected to increased IOP and injected intraocularly with vehicle (10% DMSO in PBS, 1 μl; IOP)), increased IOP and injected with NA115 (40 μM, 100 μM, and 200 μM). Blue staining represents cell nuclear staining with the retinal ganglion cells on the upper layer.

FIG. 2 shows the quantification of the images shown in FIG. 1. For each image, mean fluorescence intensity (MFI) is analyzed in the inner plexiform layer (layer between the 2 cell body layers in FIG. 1). Three frozen sections (20 μm-thick), cut through the optic disc of each eye, were collected and stained with SBDP antibody. In each section, three images were captured under 60× objective of a confocal microscope (LSM-880). For each image, MFI (mean fluorescence intensities) in the IPL layer were measured in ImageJ and averaged. N=2 animals/group.

FIG. 3 shows that compound 15 protects against retinal ganglion from increased ocular pressure-induced cell death. Immunohistochemistry is shown with staining in the peripheral area of retinal wholemounts, with anti-beta-III tubulin, a marker for retinal ganglion cells, 3 days after increased IOP in a sham animal, an animal subjected to increased IOP and injected with vehicle (10% DMSO in PBS, 1 μl; IOP)) and an animal subjected to increased IOP and injected with NA115 (200 μM). Scale bar=100 microns.

FIG. 4 shows quantification of density of anti-beta III Tubulin (retinal ganglion cell marker) positive cells in the peripheral area of retinal wholemounts of wildtype mice after IOP elevation or sham surgery. Vehicle (10% DMSO in PBS, 1 μl) or NA115 (200 μM, 1 μl) was injected 2 h after IOP elevation. Retinal whole mounts were prepared 3 days after the surgery. One-way ANOVA followed by Bonferroni test. ****p<0.0001, **p<0.01. N=8 for Sham. N=7 for IOP, IOP+NA115.

FIG. 5 shows that another C2I analog, compound 17 (a.k.a. NA117), also inhibits calpain activation following increased IOP. Same experimental procedure as in FIGS. 1-4. NA117 was injectedintraocularly at a concentration of 200 μM). One-way ANOVA followed by Bonferroni test. *p<0.05, **p<0.01. Results are means±SEM of 2 animals. Scale bar=20 microns.

FIG. 6 (includes FIGS. 6A and 6B) provides stereoisomer separation and data for Example 5.

FIG. 7 (includes FIGS. 7A and 7B), FIG. 8 (includes FIGS. 8A and 8B), and FIG. 9 (includes FIGS. 9A-9D) show results for Example 6 which follows.

### DETAILED DESCRIPTION

As discussed, in one aspect, compounds of the following Formula (I) are provided:

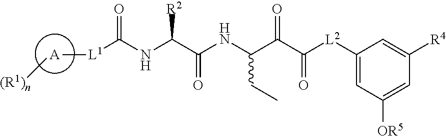

wherein A, R¹, n, L¹, R², L², R⁴ and R⁵ are as defined above. In certain aspects, preferably, R¹ is absent (n is 0 and the A ring does not contain any non-hydrogen substituents), alkyl, alkoxy or halogen, A is carbocyclic aryl such as phenyl or heteroaryl, L¹ and L² are each unsubstituted alkylene particularly methylene (—CH₂—), R⁴ is halogen such as fluoro or alkyl; and R⁵ is alkyl such as methyl; and pharmaceutically acceptable salts thereof.

Exemplary preferred A-L¹-groups include the following:

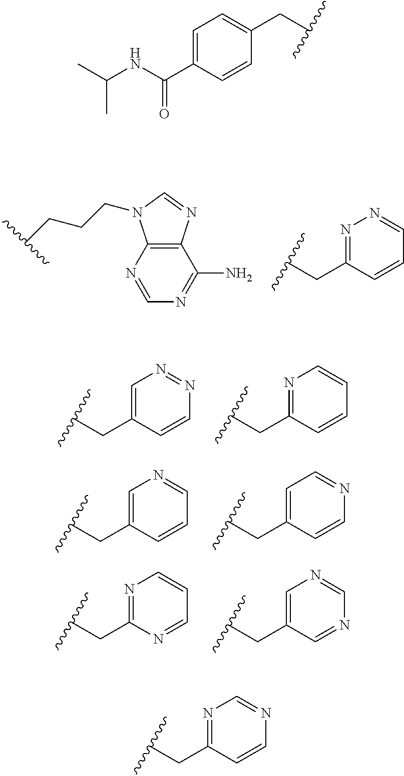

The above are also preferred A groups with other L¹ linkers.

In certain preferred aspects, the chiral carbon most adjacent L¹ has an (S) configuration. For certain aspects, the chiral carbon most adjacent to L¹ has an (R) configuration.

In certain preferred aspects, the chiral carbon most adjacent to L² has an (S) configuration. For certain aspects, the chiral carbon most adjacent to L2 has an (R) configuration

Compounds of the invention may be utilized as racemic or optically enriched mixtures.

Particularly preferred compounds of the invention are NA115, a.k.a. compound 15, and NA117, a.k.a. compound 17 as shown below.

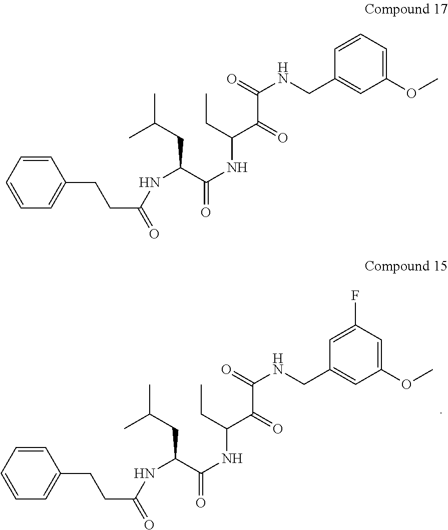 [SMILES: CCC(NC(=O)[C@H](CC(C)C)NC(=O)CCc1ccccc1)C(=O)C(=O)NCc1cccc(OC)c1 ; CCC(NC(=O)[C@H](CC(C)C)NC(=O)CCc1ccccc1)C(=O)C(=O)NCc1cc(F)cc(OC)c1]

These compounds can be calpain-2 selective inhibitors. A “calpain-2 selective inhibitor” or a “selective calpain-2 inhibitor” as referred to herein is a compound with a calpain-2 inhibition constant (Ki) lower than its Ki for calpain-1. For example, a calpain-2 selective inhibitor is a compound with a Ki for calpain-2 that is 10-fold to 100-fold lower than its Ki for calpain-1. IC₅₀ values for NA115 on the activity of calpain-1 and calpain-2 activities were measured (Wang et al., 2014). The selectivity of NA115 for calpain-2, measured as a ratio of IC50 calpain-1/IC50 calpain-2 was 31.7. The selectivity of NA117 was 24.1. [0016] Pharmaceutical compositions of the invention comprise NA115 and NA117, and a pharmaceutically acceptable excipient. Excipients used in pharmaceutical composition of the invention are safe and provide the appropriate delivery for the desired route of administration, of an effective amount of NA115 and NA117.

Compounds of the invention possess asymmetric carbon atoms (optical or chiral centers); the enantiomers, racemates, stereoisometric forms that may be defined, in terms of absolute stereochemistry, as (R)-or (S)-isomers, and individual isomers are encompassed within the scope of the present invention. The present invention is meant to include compounds in racemic and optically pure forms as discussed above. Optically active (R)- and (S)-isomers maybe prepared using chiral synthons or chiral reagents, or resolved using conventional techniques.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

“Alkyl” refers to a saturated, straight or branched hydrocarbon chain radical consisting solely of carbon and hydrogen atoms, having from one to twelve carbon atoms (C₁-C₁₂ alkyl), from one to eight carbon atoms (C₁-C₈ alkyl) or from one to six carbon atoms (C₁-C₆ alkyl), and which is attached to the rest of the molecule by a single bond. Exemplary alkyl groups include methyl, ethyl, n-propyl, 1-methylethyl (iso-propyl), n-butyl, n-pentyl, 1,1-dimethylethyl (t-butyl), 3-methylhexyl, 2-methylhexyl, and the like.

“Alkylene” or “alkylene chain” refers to a straight or branched divalent hydrocarbon (alkyl) chain linking the rest of the molecule to a radical group, consisting solely of carbon and hydrogen, respectively. Alkylenes can have from one to twelve carbon atoms, e.g., methylene, ethylene, propylene, n-butylene, and the like. The alkylene chain is attached to the rest of the molecule through a single or double bond. The points of attachment of the alkylene chain to the rest of the molecule can be through one carbon or any two carbons within the chain. “Optionally substituted alkylene” refers to alkylene or substituted alkylene.

“Alkoxy” refers to a radical of the formula —ORₐ where Rₐ is an alkyl having the indicated number of carbon atoms as defined above. Examples of alkoxy groups include without limitation —O-methyl (methoxy), —O-ethyl (ethoxy), —O-propyl (propoxy), —O-isopropyl (iso propoxy) and the like.

“Carbocyclic aryl” refers to a hydrocarbon ring system radical comprising hydrogen, 6 to 18 carbon atoms and at least one aromatic ring, but without any hetero (N, O or S) ring members in the aromatic ring. Exemplary carbocyclic aryls are hydrocarbon ring system radical comprising hydrogen and 6 to 9 carbon atoms and at least one aromatic ring; hydrocarbon ring system radical comprising hydrogen and 9 to 12 carbon atoms and at least one aromatic ring; hydrocarbon ring system radical comprising hydrogen and 12 to 15 carbon atoms and at least one aromatic ring; or hydrocarbon ring system radical comprising hydrogen and 15 to 18 carbon atoms and at least one aromatic ring. For purposes of this invention, the carbocyclic aryl radical may be a monocyclic, bicyclic, tricyclic or tetracyclic ring system, which may include fused or bridged ring systems. Carbocyclic aryl radicals include, but are not limited to, carbocyclic aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. “Optionally substituted carbocyclic aryl” refers to an unsubstituted carbocyclic aryl group or a substituted carbocylic aryl group.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a stable 5-12 membered ring, a stable 5-10 membered ring, a stable 5-9 membered ring, a stable 5-8 membered ring, a stable 5-7 membered ring, or a stable 6 membered ring that comprises at least 1 heteroatom, at least 2 heteroatoms, at least 3 heteroatoms, at least 4 heteroatoms, at least 5 heteroatoms or at least 6 heteroatoms. Heteroaryls may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. The heteroatom may be a member of an aromatic or non-aromatic ring, provided at least one ring in the heteroaryl is aromatic. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl).

Various compounds and substituents that are “optionally substituted” may be suitably substituted at one or more available positions by e.g. halogen (F, Cl, Br, I); nitro; hydroxy; amino; alkyl such as C₁₋₄alkyl; alkenyl such as C₂₋₈alkenyl; alkoxy e.g. C1-6alkxoy, alkylamino such as C₁₋₈ alkylamino; carbocyclic aryl such as phenyl, naphthyl, anthracenyl, etc; heteroaryl; and the like.

A compound of the invention, as described above, can be formulated as a pharmaceutical dosage form and administered to a subject in need of treatment, for example, a mammal, such as a human patient, in a variety of forms adapted to the chosen route of administration. The compositions of the present invention may be administered in a variety of different ways, including topically and by intraocular injection, intraocular perfusion, periocular injection or retrobulbar (sub-tenon) injection. Compounds of the present invention may be contained in various types of ophthalmic compositions, in accordance with formulation techniques known to those skilled in the art. For example, the compounds may be included in solutions, suspensions and other dosage forms adapted for topical, intravitreal or intracameral use.

Solutions of the compounds of the invention can be prepared in water or a physiologically acceptable buffer, optionally mixed with a nontoxic surfactant, including cyclodextrins. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the compounds of the invention which are adapted for the extemporaneous preparation of sterile injectable or infusible solutions or dispersions. In all cases, the ultimate dosage form should be sterile, fluid and stable under the conditions of manufacture and storage. The liquid carrier can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the compounds of the invention in the required amount in the appropriate solvent with various of the other ingredients enumerated above, as required, followed by filter sterilization. In the case of sterile powders for the preparation of sterile injectable solutions, the preferred methods of preparation are vacuum drying and freeze-drying techniques, which yield a powder of the active ingredient plus any additional desired ingredient present in the previously sterile-filtered solutions.

Useful dosages of compounds of the invention can be determined by comparing their in vitro activity, and in vivo activity in animal models. Methods for the extrapolation of effective dosages in mice, and other animals, to humans are known to the art; for example, see U.S. Pat. No. 4,938,949. The amount of the compounds of the invention required for use in treatment will vary depending on the particular therapeutic agent, the composition, if there is one, that comprises the therapeutic agent, the route of administration, the nature of the condition being treated and the age and condition of the patient, and will be ultimately at the discretion of the attendant physician or clinician.

A therapeutically effective dose can be determined empirically, by conventional procedures known to those of skill in the art. See, e.g., The Pharmacological Basis of Therapeutics, Goodman and Gilman, eds., Macmillan Publishing Co., New York. For example, an effective dose can be estimated initially either in cell culture assays or in suitable animal models. The animal model may also be used to determine the appropriate concentration ranges and routes of administration. Such information can then be used to determine useful doses and routes for administration in humans. A therapeutic dose can also be selected by analogy to dosages for comparable therapeutic agents.

The particular mode of administration and the dosage regimen will be selected by the attending clinician, considering the particulars of the case (e.g., the subject, the disease, the disease state involved, and whether the treatment is prophylactic). Treatment may involve daily or multi-daily doses of compound(s) over a period of a few days to months, or even years.

### EXAMPLES

### Example 1: Synthesis of Intermediates for Compounds NA115 and NA117

Step 1: Preparation of tert-butyl (1-hydroxybutan-2-yl)carbamate. 2-aminobutan-1-ol (1 g) was dissolved in chloroform (50 mL) and treated with di-tert-butyl dicarbonate (2.5 g) and sodium hydroxide solution (20 mL, 2M). After stirring overnight at room temperature, the solvents were removed and the residue purified by flash chromatography (hexane/ethyl acetate 0-50%) to afford tert-butyl (1-hydroxybutan-2-yl)carbamate (1.83 g, 86% yield).

Step 2: Preparation of tert-butyl (1-oxobutan-2-yl)carbamate. DMSO (2.34 g, 3 equiv) was added to a stirred solution of (ClCO)2 (1.9 g, 1.5 equiv) in CH2Cl2 (20 mL) at −78° C. After stirring for 10 min, tert-butyl (1-hydroxybutan-2-yl)carbamate (1.838 g) in CH2Cl2 (10 mL) was added dropwise and the resulting mixture was allowed to stir for 30 min. Et3N (4.04 g, 4 equiv) was then added and the reaction mixture was allowed to warm to room temperature and stirred for a further 30 min. Water (20 mL) was then added, the reaction mixture was extracted with CH2Cl2 (3×10 mL), and the combined organic extracts were dried and concentrated in vacuo to give a residue which was purified by flash chromatography (hexane/ethyl acetate 0-20%) to afford tert-butyl (1-oxobutan-2-yl)carbamate (1.57% g, 86% yield).

Step 3: Preparation of tert-butyl (1-cyano-1-hydroxybutan-2-yl)carbamate. Tert-butyl (1-oxobutan-2-yl)carbamate (18.9 g) was dissolved in dioxane (400 mL) and chilled to 0° C. for 10 min, at which time NaHS03 (52.64 g) in water (200 mL) was added. The reaction mixture was allowed to stir at 0° C. for 10 min and KCN (26.22 g) in water (200 mL) was added and the solution was stirred overnight.

The reaction mixture was worked up by diluting with ethyl acetate (2000 mL) and washing the organic layer with three portions of saturated sodium bicarbonate. The organic layer was dried over sodium sulfate, filtered and concentrated to dryness to yield tert-butyl (1-cyano-1-hydroxybutan-2-yl)carbamate (24.62 g).

Step 4: Preparation of methyl 3-amino-2-hydroxypentanoate. Tert-butyl (1-cyano-1-hydroxybutan-2-yl)carbamate (24.62 g) in HCl/MeOH (˜500 mL) (prepared from 400 of methanol and 180 mL of AcCl) was heated at reflux for 25 h. The solution was evaporated and the crude methyl 3-amino-2-hydroxypentanoate was used without further purification.

Step 5: Preparation of methyl 3-((S)-2-((tert-butoxycarbonyl)amino)-4-methylpentanamido)-2-hydroxypentanoate. The crude methyl 3-amino-2-hydroxypentanoate HCl salt (˜5.29 mmol theoretical) was dissolved in acetonitrile (50 mL) and treated with triethylamine (2 mL), HATU (2.2 g) followed by BOC-leucine hydrate (1.318 g) and the mixture stirred overnight at room temperature. The product was purified by flash chromatography (hexane/EtOAc, 0 to 30%) gave a crude mixture of 4 diastereomers.

Step 6: Preparation of 3-((S)-2-((tert-butoxycarbonyl)amino)-4-methylpentanamido)-2-hydroxypentanoic acid (Intermediate A). Methyl 3-((S)-2-((tert-butoxycarbonyl)amino)-4-methylpentanamido)-2-hydroxypentanoate (2.632 g) was dissolved in a mixture of 1M NaOH (8 ml) and THF (8 ml) overnight at which time the solution was partitioned between ethyl acetate and dilute HCL, extracted with ethyl acetate (2×), the combined extracts dried, filtered and evaporated to dryness to afford the crude 3-((S)-2-((tert-butoxycarbonyl)amino)-4-methylpentanamido)-2-hydroxypentanoic acid (2.25 g, ˜89% yield).

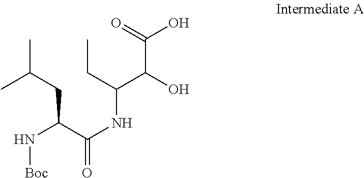 [SMILES: CCC(NC(=O)[C@H](CC(C)C)NC(=O)OC(C)(C)C)C(O)C(=O)O]

Preparation of (2S)-2-amino-N-(1-((3-methoxybenzyl)amino)-1,2-dioxopentan-3-yl)-4-methylpentanamide: Intermediate 2.B. 3-((S)-2-((tert-butoxycarbonyl)amino)-4-methylpentanamido)-2-hydroxypentanoic acid (2.24 g, 6.47 mmol) was treated with 3-methoxybenzylamine (0.976 g, 7.12 mmol), HATU (2.95 g, 7.76 mmol), and DIPEA (1.255 g, 9.71 mmol) in ACN (50 mL) and stirred overnight at room temperature. After removal of the solvent at reduced pressure, the product was purified byflash chromatography (hexane-ethyl acetate, 0-100% to afford to afford tert-butyl ((2S)-1-((1-((3-methoxybenzyl)amino)-1,2-dioxopentan-3-yl)amino)-4-methyl-1-oxopentan-2-yl)carbamate which was dissolved in 4N HCl in dioxane (50 mL) and stirred at room temperature for 30 min. Removal of the solvent followed by drying in vacuo afforded pure (2S)-2-amino-N-(1-((3-methoxybenzyl)amino)-1,2-dioxopentan-3-yl)-4-methylpentanamide as the hydrochloride salt (2.15 g, 83% yield).

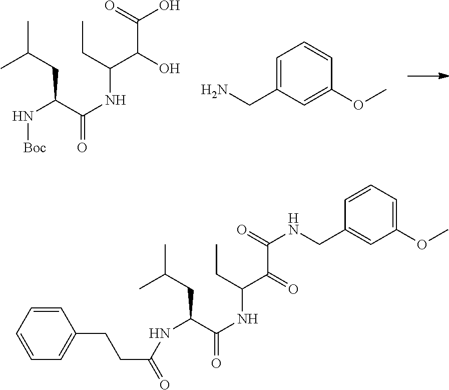

Preparation of (2S)-2-amino-N-(1-((3-fluoro-5-methoxybenzyl)amino)-2-hydroxy-1-oxopentan-3-1)-4-methylpentanamide: Intermediate 3.B. 3-((S)-2-((tert-butoxycarbonyl)amino)-4-methylpentanamido)-2-hydroxypentanoic acid (2.00 g, 5.78 mmol) was treated with 3-methoxy-5-fluorobenzylamine (0.986 g, 6.36 mmol), HATU (2.64 g, 6.94 mmol), and DIPEA (1.12 g, 8.67 mmol) in acetonitrile (40 mL) and stirred overnight at room temperature. After removal of the solvent at reduced pressure, the product was purified by flash chromatography (hexane-ethyl acetate, 0-100% to afford to afford tert-butyl ((2S)-1-((1-((3-fluoro-5-methoxybenzyl)amino)-2-hydroxy-1-oxopentan-3-yl)amino)-4-methyl-1-oxopentan-2-yl)carbamate which was dissolved in 4N HCl in dioxane (50 mL) and stirred at room temperature for 30 min. Removal of the solvent followed by drying in vacuo afforded pure (2S)-2-amino-N-(1-((3-fluoro-5-methoxybenzyl)amino)-2-hydroxy-1-oxopentan-3-yl)-4-methylpentanamide as the hydrochloride salt (2.34 g, 96%).

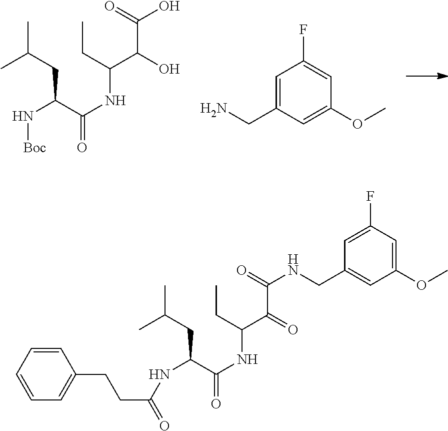 [SMILES: CCC(NC(=O)[C@H](CC(C)C)NC(=O)OC(C)(C)C)C(O)C(=O)O ; COc1cc(F)cc(CN)c1 ; CCC(NC(=O)[C@H](CC(C)C)NC(=O)CCc1ccccc1)C(=O)C(=O)NCc1cc(F)cc(OC)c1]

### Example 2: Synthesis of NA117

Preparation of N-(3-methoxybenzyl)-3-((S)-4-methyl-2-(3-phenylpropanamido)pentanamido)-2-oxopentanamide (Compound 2.3) (Compound 17) (2S)-2-amino-N-(1-((3-methoxybenzyl)amino)-1,2-dioxopentan-3-yl)-4-methylpentanamide hydrochloride (Intermediate 2.B) (50 mg) was dissolved/suspended in acetonitrile (1 mL), and treated with 3-phenylpropanoic acid (1.1 equiv), HATU (1.2 equiv) and DIPEA (2.5 equiv) and stirred at room temperature until LCMS analysis indicated completion of reaction. Evaporation of the solvents, followed by partition between water and ethyl acetate gave a residue which was purified by flash chromatography to afford the corresponding amide. This material (1 equiv) was dissolved in dichloromethane (25 mL/mmol) and treated with Dess-Martin periodinane (DMP) (2 equiv) stirring at room temperature for 2 h at which time the reaction mixture was partitioned between saturated bicarbonate solution and ethyl acetate. The aqueous layer was extracted twice more with ethyl acetate and the combined organic layers were washed with water, dried filtered, and concentrated to dryness. The residue was then purified by preparative HPLC to afford the pure N-(3-methoxybenzyl)-3-((S)-4-methyl-2-(3-phenylpropanamido)--pentan--amido)-2-oxo pentamide (22.4 mg).

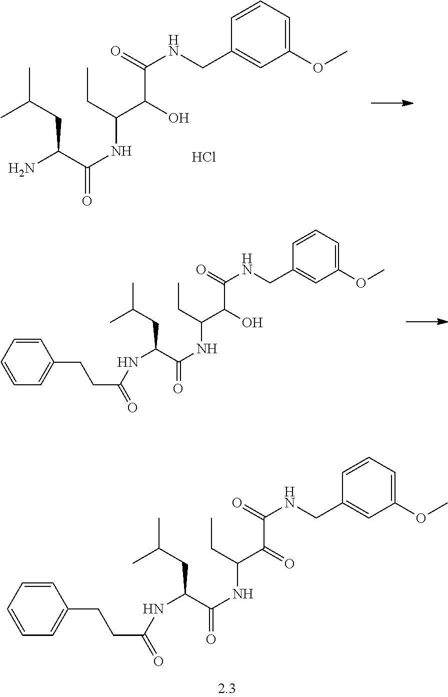 [SMILES: CCC(NC(=O)[C@@H](N)CC(C)C)C(O)C(=O)NCc1cccc(OC)c1 ; Cl ; CCC(NC(=O)[C@H](CC(C)C)NC(=O)CCc1ccccc1)C(O)C(=O)NCc1cccc(OC)c1 ; CCC(NC(=O)[C@H](CC(C)C)NC(=O)CCc1ccccc1)C(=O)C(=O)NCc1cccc(OC)c1]

### Example 3: Synthesis of NA115

Preparation of N-(3-fluoro-5-methoxybenzyl)-3-((S)-4-methyl-2-(3-phenylpropanamido)-pentanamido)-2-oxopentanamide (Compound 3.3) (Compound 15) (2S)-2-amino-N-(1-((3-fluoro-5-methoxybenzyl)amino)-1,2-dioxopentan-3-yl)-4-methylpentanamide hydrochloride (Intermediate 3.B) (50 mg) was dissolved/suspended in acetonitrile (1 mL), and treated with 3-phenylpropanoic acid (1.1 equiv), HATU (1.2 equiv) and DIPEA (2.5 equiv) and stirred at room temperature until LCMS analysis indicated completion of reaction. Evaporation of the solvents, followed by partition between water and ethyl acetate gave a residue which was purified by flash chromatography to afford the corresponding amide. This material (1 equiv) was dissolved in dichloromethane (25 mL/mmol) and treated with Dess-Martin periodinane (DMP) (2 equiv) stirring at room temperature for 2 h at which time the reaction mixture was partitioned between saturated bicarbonate solution and ethyl acetate. The aqueous layer was extracted twice more with ethyl acetate and the combined organic layers were washed with water, dried filtered, and concentrated to dryness. The residue was then purified by preparative HPLC to afford the pure N-(3-fluoro-5-methoxybenzyl)-3-((S)-4-methyl-2-(3-phenylpropanamido)--pentanamido)-2-oxopentanamide (6.5 mg).

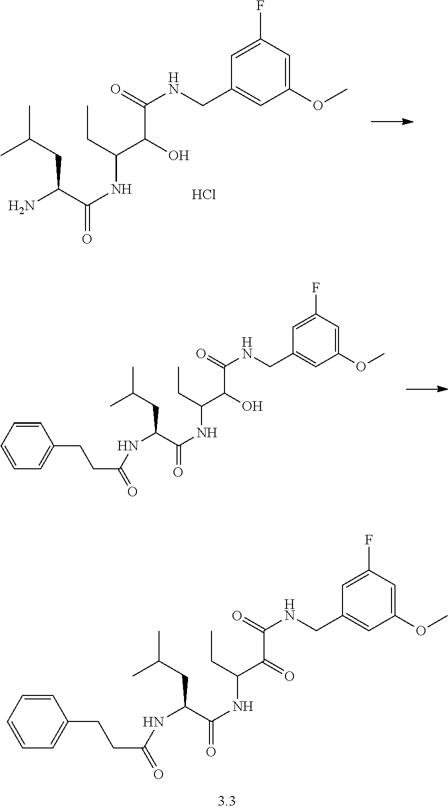 [SMILES: CCC(NC(=O)[C@@H](N)CC(C)C)C(O)C(=O)NCc1cc(F)cc(OC)c1 ; Cl ; CCC(NC(=O)[C@H](CC(C)C)NC(=O)CCc1ccccc1)C(O)C(=O)NCc1cc(F)cc(OC)c1 ; CCC(NC(=O)[C@H](CC(C)C)NC(=O)CCc1ccccc1)C(=O)C(=O)NCc1cc(F)cc(OC)c1]

### Example 4: Testing of Compounds NA115 and NA117 in Mice

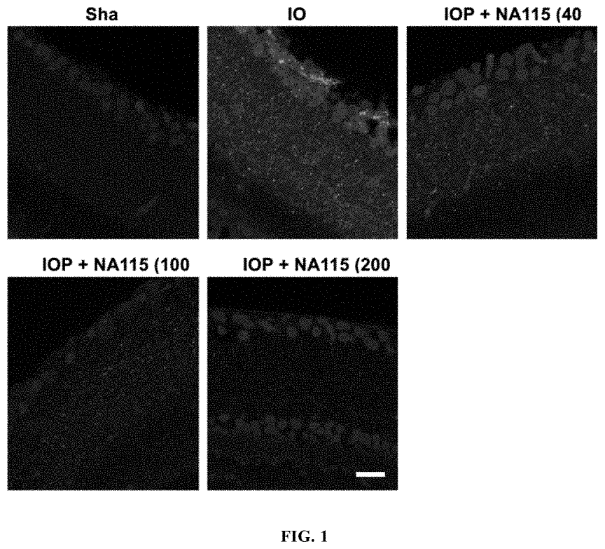

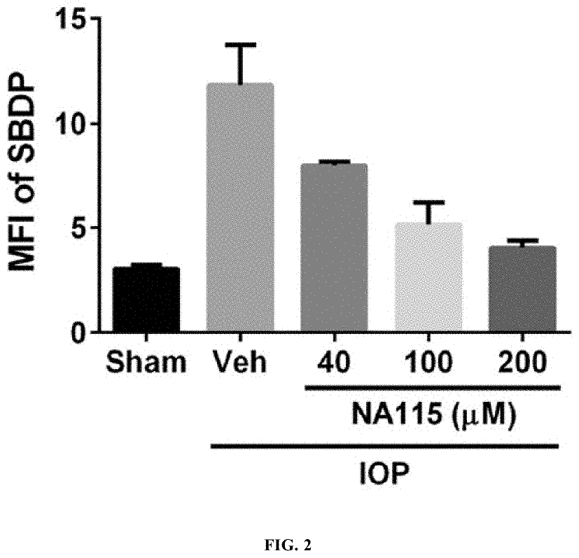

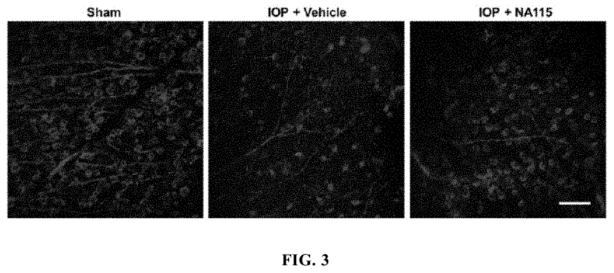

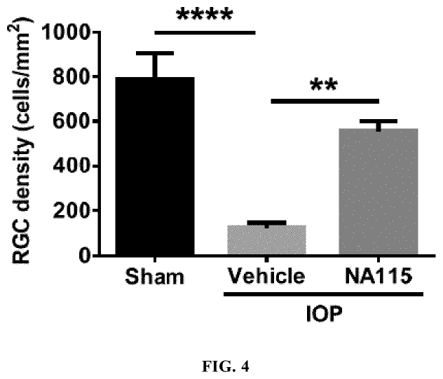

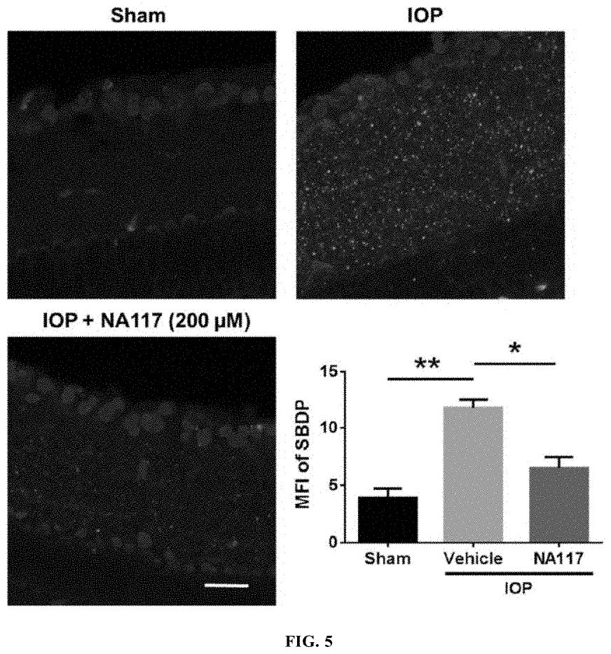

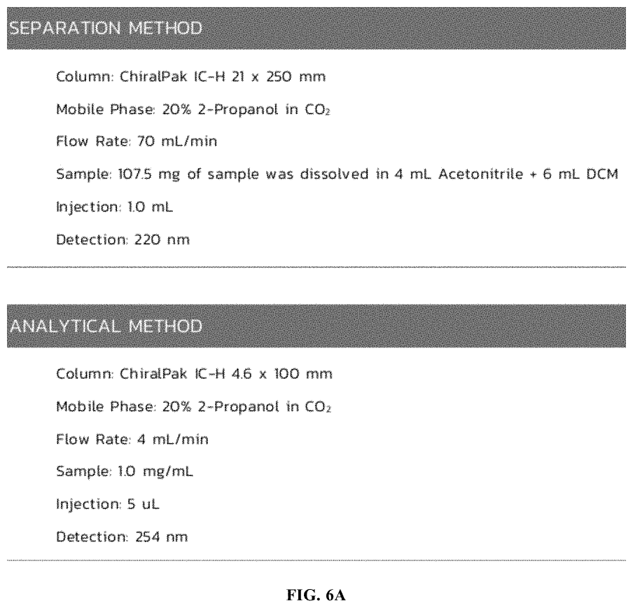

The model of acute glaucoma previously reported in Wang et al. (2016) was used. In this model, intraocular pressure (IOP) was increased to 110 mm Hg for 1 h with the mouse under anesthesia. Two h later mice received an intraocular injection of various concentrations of a calpain-2 inhibitor and were returned to their home cages. They were sacrificed 4 h later for determination of calpain activity using immunohistochemistry to stain for the spectrin breakdown product (SBDP) selectively generated by calpain-mediated truncation of spectrin. Previous studies (Wang et al., 2016) have shown that, at this time-point calpain activity represents calpain-2 activity. Other groups of mice were sacrificed 3 days after increase in IOP for the analysis of the number of retinal ganglion cells. This was done by immunohistochemistry in retina whole mounts to stain for beta-III tubulin, a retinal ganglion cell marker. The results are presented in FIG. 1-5.

### Example 5: Separation of Isomers

There are 2 chiral centers for NA115. NA115A, where chiral center 1 is the S-S form (Compound 15(S), or NA115A) and chiral center 2 is the S- form was separated from the S-R-form (Compound 15(R), or NA115B) using methods that are well-known methods for separating diastereoisomers.

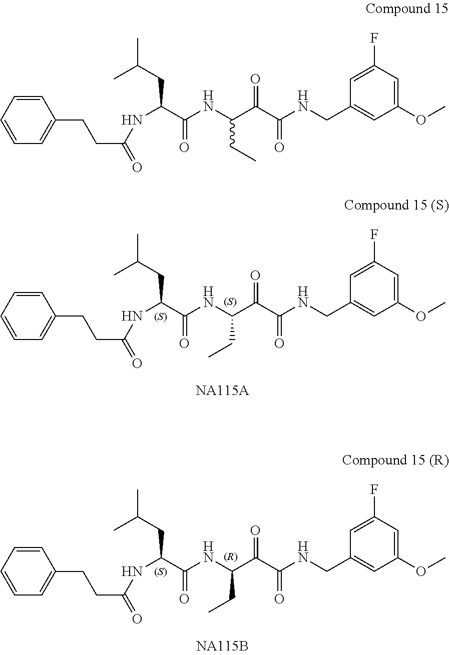

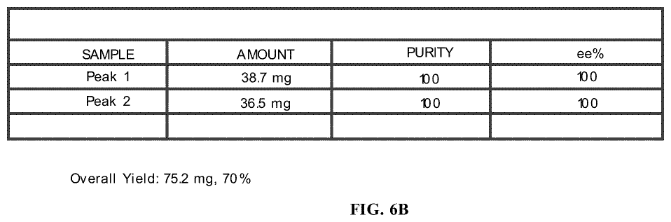

Separation reports including an exemplary procedure and results therefrom are shown in FIGS. 6A-6B.

NA115A (Compound 15 (S above) was introduced at various concentrations into an in vitro mix comprising succinic-Leu-Tyr-AMC and human calpain-1 or calpain-2 (Sasaki et al, 1984), and the kinetics of the loss of fluorescence were determined for each of the calpains. The Kis of NA115, NA115A and NA115B for calpain-1 and calpain-2 are shown in Tables 1-2 below. The efficacy of NA115 against calpain-1 or calpain-2 appears to be only in NA115A.

TABLE 1

| | | | Ratio KiCalpain- |
|---|---|---|---|
| Calpain-2 | IC50 | Ki | 1/KiCalpain-2 |
| NA115 | 170 nM | 103 nM | 4.5 |
| NA115A | 196 nM | 124 nM | 1.5 |
| NA115B | >10 μM | >10 μM | N/A |

TABLE 2

| Calpain-1 | IC50 | Ki |
|---|---|---|
| NA115 | 750 nM | 470 nM |
| NA115A | 331 nM | 189 nM |
| NA115B | >10 μM | >10 μM |

### Example 6: Epimerization of NA115 in Pig Vitreous Fluid

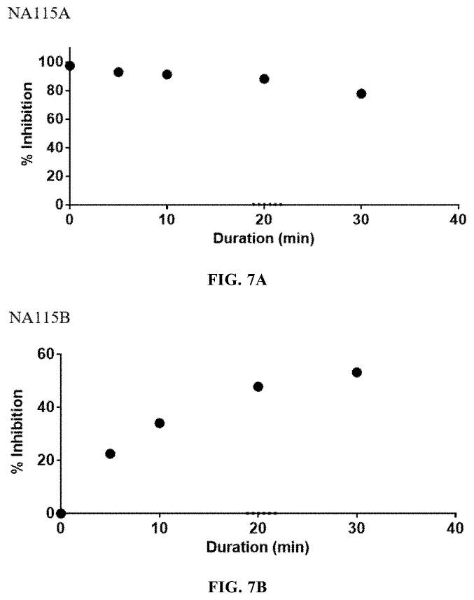

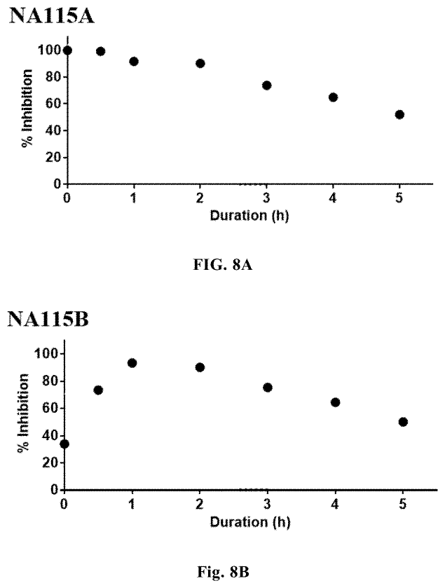

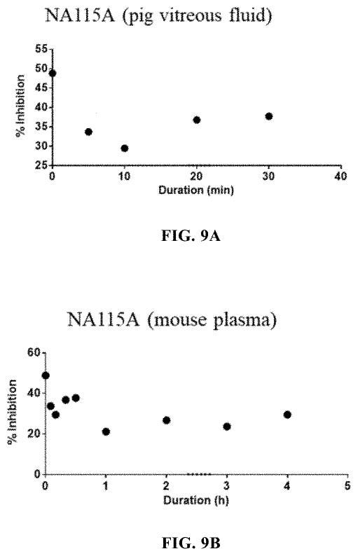

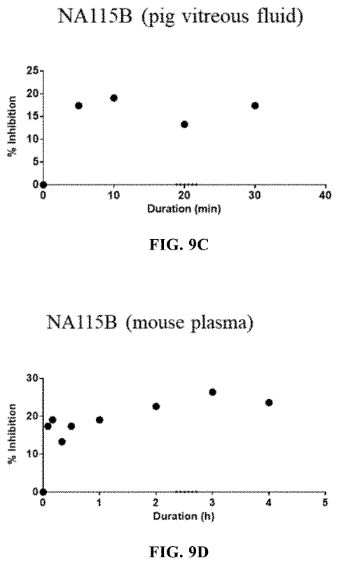

NA115A or NA115B (2 μM) was incubated with pig vitreous fluid for various periods of time at 35° C. Aliquots were then tested in the calpain-2 assay. Results show that there is rapid decrease in the inhibitory effect of NA115A accompanied by an increased inhibitory effect of NA115B. These results suggest that there is rapid epimerization of NA115A/B (FIGS. 7A and 7B). These results were confirmed in mouse plasma. In addition, the inhibition results at final concentration of NA115A or NA115B in the incubation of 2 μM are shown in FIGS. 8A and 8B. These results were replicated at a lower concentration of NA115A and NA115B, closer to the IC₅₀ against calpain-2 (200 nM). (FIGS. 9A-9D).

These results show that rapid epimerization of the S-S and S-R diastereoisomers and a slower metabolism of the molecule, which results in loss of inhibitory activity. This was further studied by determining the stability of the racemate mixture in mouse plasma.

### Example 7: Plasma Stability of NA115 (Powerpoint File Attached: Stability NA115.pptx)

Stability of NA115 was evaluated with NA115 solubilized in 2-Hydroxypropyl)-beta-cyclodextrin or in captisol. These results confirm that the molecule is degraded in mouse plasma with a half-life between 9 and 15 h depending on the solvent.

Moreover, 1 mM of the NA115 in beta-cyclodextrin was diluted 5 times in freshly prepared mouse plasma (200 μM NA115 in plasma). The mixture was incubated at 37 degree. At indicated time point, 1 μl of the mixture was added to 99 ul of calpain assay solution containing 5 mM Ca2+, 200 μM Suc-Leu-Tyr-AMC substrate and 100 nM calpain-2. Hydrolysis rate was monitored in the plate reader. As a control, 1 μl of plasma alone was subjected to calpain assay and its hydrolysis rate was set as 100% of calpain activity.

1. A compound of the following Formula (I) 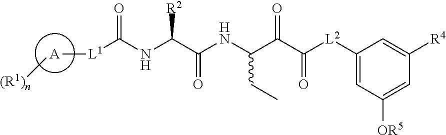
wherein A is carbocyclic aryl, or heteroaryl
R¹ is a non-hydrogen substituent;
n is an integer of from 0 (where the ring A is unsubstituted) to the value permitted by the valence of A;
L¹ and L² are each the same or different optionally substituted alkylene having 1 to 6 carbons;
R² is non-hydrogen substituent,
R⁴ is hydrogen or halogen such as fluoro; R⁵ is C₁₋₆alkyl such as methyl; and pharmaceutically acceptable salts thereof.

2. A compound of claim 1 wherein A is phenyl.

3. A compound of claim 1, wherein L1 and L2 each is —CH₂—.

4. A compound of claim 1 wherein R⁴ is fluoro and R⁵ is methyl.

5. A compound that is compound 17 having the following structure:

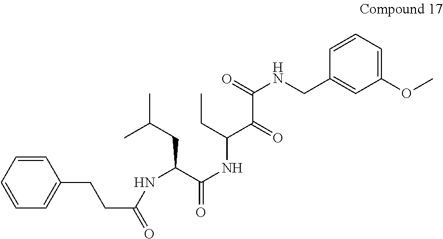 [SMILES: CCC(NC(=O)[C@H](CC(C)C)NC(=O)CCc1ccccc1)C(=O)C(=O)NCc1cccc(OC)c1]

6. A compound that is compound 15 of the following structure:

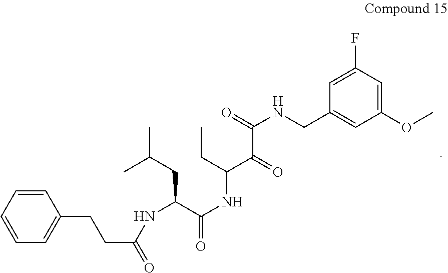 [SMILES: CCC(NC(=O)[C@H](CC(C)C)NC(=O)CCc1ccccc1)C(=O)C(=O)NCc1cc(F)cc(OC)c1]

7. A compound of claim 1 wherein the compound is a racemate.

8. A compound of claim 1 wherein the compound is present as an optically enriched mixture.

9. A pharmaceutical composition comprising the compound of claim 1 and a pharmaceutically acceptable excipient.

10. A method for treating glaucoma-related nerve damage in a patient, said method comprising administering to a patient in need thereof an effective amount of a compound or composition of any one of claims 1 through 9.

11. A method of treating a subject suffering from an eye disorder, comprising:
administering to the subject an effective amount of a compound or composition of claim 1.

12. The method of claim 11 wherein the eye disorder is associated with retinal neuronal cell death.

13. The method of claim 11 wherein the subject is suffering from glaucoma.

14. The method of claim 1 wherein the compound or composition is administered via a method selected from the group consisting of intravitreal injection, intraocular injection, intraocular perfusion, periocular injection and sub-Tenon injection.
